(19) United States
(12) Patent Application Publication
(10) Pub. No.: US 2026/0057999 A1
(43) Pub. Date: Feb. 26, 2026

(54) ASSISTANCE METHOD, ASSISTANCE PROGRAM, ASSISTANCE DEVICE, AND ASSISTANCE SYSTEM FOR ASSISTING PREHABILITATION

(71) Applicants: KABUSHIKI KAISHA YAKULT HONSHA, Tokyo (JP); NATIONAL UNIVERSITY CORPORATION TOKAI NATIONAL HIGHER EDUCATION AND RESEARCH SYSTEM, Nagoya-shi, Aichi (JP)

(72) Inventors: Naoki HAYASHI, Tokyo (JP); Ryoya YANO, Tokyo (JP); Ken SHIMOTORI, Tokyo (JP); Shogo HAYAMA, Tokyo (JP); Yukihiro YOKOYAMA, Nagoya-shi, Aichi (JP)

(21) Appl. No.: 19/104,795

(22) PCT Filed: Oct. 27, 2023
(86) PCT No.: PCT/JP2023/038895

§ 371 Date: Feb. 19, 2025

(30) Foreign Application Priority Data
Oct. 31, 2022 (JP) 2022-174127

(51) Int. Cl. G16H 20/60

(52) U.S. Cl. CPC G16H 20/60

(57) ABSTRACT

An assistance device 3 for assisting prehabilitation for a patient who is scheduled to undergo surgery, the assistance device comprising an acquisition unit 31 for acquiring preoperative information D1 on the patient's condition during a specified period before surgery, the preoperative information D1 including at least one of nutrition consumed by the patient and the patient's strength training status, and the nutrition containing at least one of amino acids, probiotics, and prebiotics.

### TECHNICAL FIELD

The present invention relates to a prehabilitation assistance technology.

### BACKGROUND ART

The postoperative course (length of hospital stay, postoperative complications, etc.) of a patient also depends on the patient's condition before surgery. In recent years, prehabilitation, which involves providing patients with exercise and health management before surgery in order to improve their postoperative course, has been attracting attention.

For example, NPL 1 indicates that the nutritional status of patients before surgery, physical function through exercise, and anxiety and stress (mental state) before surgery affect their postoperative course.

### CITATION LIST

### Non-Patent Literature

NPL 1: “Recent topic in anesthesiology field: Recommendations for prehabilitation in cancer surgery,” Kakehashi, Hyogo Cancer Center, March 2022, vol. 80, pp. 2-3, internet <URL: https://hyogo-cc.jp/general/pdf/no80.pdf>

### SUMMARY OF INVENTION

### Technical Problem

In NPL 1, the nutritional status before surgery was evaluated based on BMI, body weight, and food intake, but was not evaluated based on the type of nutrition. However, the present inventors' research has revealed that the type of nutrition also affects the postoperative course. With conventional prehabilitation, it is difficult to appropriately evaluate the postoperative prognosis risk. In addition, patients before surgery often wait at home rather than being hospitalized, and completion of the prehabilitation program is left up to each individual. Accordingly, problems such as variation in program compliance rates and reduced motivation to complete the program have been pointed out. These problems also make it difficult to determine the postoperative prognostic risk.

The present invention was made to solve the above problems, and an object thereof is to appropriately assist prehabilitation.

### Solution to Problem

In order to achieve the above object, the present invention includes the following aspects.

### Item 1.

An assistance method using a computer for assisting prehabilitation for a patient who is scheduled to undergo surgery,
the method comprising an acquisition step of acquiring preoperative information on the patient's condition during a specified period before surgery,
the preoperative information including at least one of nutrition consumed by the patient and the patient's strength training status, and
the nutrition containing at least one of amino acids, probiotics, and prebiotics.

### Item 2.

The assistance method according to Item 1, wherein the nutrition contains at least one of amino acids and synbiotics.

### Item 3.

The assistance method according to Item 1 or 2, wherein the preoperative information includes the patient's bowel movements.

### Item 4.

The assistance method according to any one of Items 1 to 3, wherein the preoperative information includes the patient's activity level.

### Item 5.

The assistance method according to Item 4, wherein the activity level includes the patient's steps and calories burned.

### Item 6.

The assistance method according to any one of Items 1 to 5, further comprising a prediction step of predicting the patient's postoperative course based on the preoperative information.

### Item 7.

The assistance method according to any one of Items 1 to 6, further comprising, before the acquisition step:
a recording step of recording the preoperative information using a patient terminal managed by the patient; and
a transmission step of transmitting the preoperative information from the patient terminal to the computer.

### Item 8.

An assistance program for assisting prehabilitation for a patient who is scheduled to undergo surgery,
the assistance program causing a computer to execute an acquisition step of acquiring preoperative information on the patient's condition during a specified period before surgery,
the preoperative information including at least one of nutrition consumed by the patient and the patient's strength training status, and
the nutrition containing at least one of amino acids, probiotics, and prebiotics.

### Item 9.

An assistance device for assisting prehabilitation for a patient who is scheduled to undergo surgery,
the assistance device comprising an acquisition unit for acquiring preoperative information on the patient's condition during a specified period before surgery,
the preoperative information including at least one of nutrition consumed by the patient and the patient's strength training status, and
the nutrition containing at least one of amino acids, probiotics, and prebiotics.

### Item 10.

An assistance system comprising:
the assistance device according to Item 9; and
a patient terminal communicably connected to the assistance device and recording the preoperative information.

### Advantageous Effects of Invention

The present invention can appropriately assist prehabilitation.

### BRIEF DESCRIPTION OF DRAWINGS

FIG. 1 is a block diagram showing a schematic configuration of an assistance system according to an embodiment of the present invention.

FIG. 2 shows an example of the home screen of a recording application.

FIGS. 3 (a) and (b) show examples of the screen for recording the patient's strength training status.

FIGS. 4 (a) and (b) show examples of the screen for recording the patient's strength training status.

FIG. 5 shows an example of the screen for recording the patient's strength training status.

FIGS. 6 (a) and (b) show examples of the screen for recording the patient's bowel movements.

FIGS. 7 (a) and (b) show examples of the screen for recording nutrition taken by the patient.

FIG. 8 shows an example of the screen for recording the patient's activity level.

FIG. 9 shows an example of preoperative information displayed on the display of a medical personnel terminal.

FIG. 10 shows an example of a patient list screen displayed on the display of a medical personnel terminal.

FIG. 11 is a flowchart showing the processing steps of a method for assisting prehabilitation for a patient.

### DESCRIPTION OF EMBODIMENTS

An embodiment of the present invention is described below with reference to the attached drawings. The present invention is not limited to the following embodiment, and various modifications are possible without departing from the spirit of the invention.

### Overall Configuration

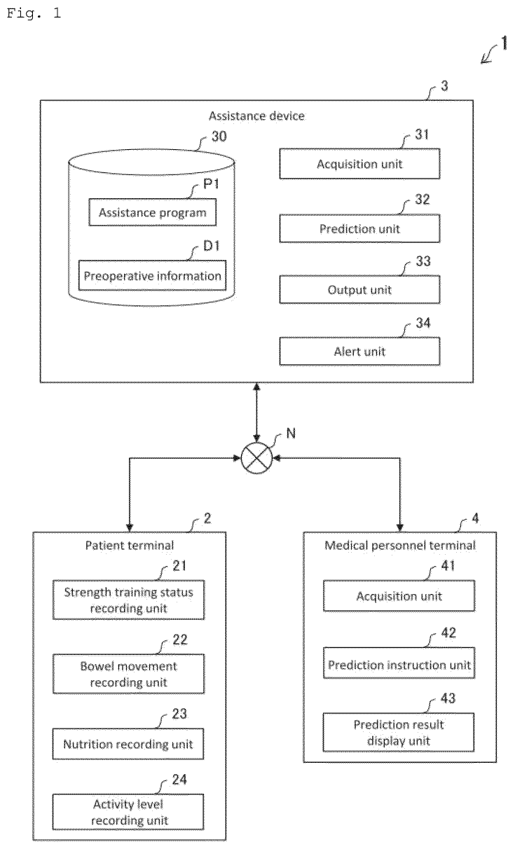

FIG. 1 is a block diagram showing a schematic configuration of an assistance system 1 according to an embodiment of the present invention. The assistance system 1 is a system for assisting prehabilitation for a patient who is scheduled to undergo surgery, and comprises a patient terminal 2, an assistance device 3, and a medical personnel terminal 4. The patient terminal 2, the assistance device 3, and the medical personnel terminal 4 are communicably connected via a network N, such as the internet.

### Patient Terminal

The patient terminal 2 is a terminal managed by the patient and is a mobile device, such as a smartphone. The patient terminal 2 may be owned by the patient or may be loaned to the patient, as long as it is something that the patient can use on a daily basis for a predetermined period (e.g., several months) before surgery.

The patient can use the patient terminal 2 to record the patient's preoperative information during a specified period before surgery. In this embodiment, the preoperative information includes the patient's strength training status, the patient's bowel movements, nutrition taken by the patient, and the patient's activity level. A recording application for recording the preoperative information is installed on the patient terminal 2. By executing the recording application, the patient terminal 2 realizes the following function blocks: a strength training status recording unit 21, a bowel movement recording unit 22, a nutrition recording unit 23, and an activity level recording unit 24.

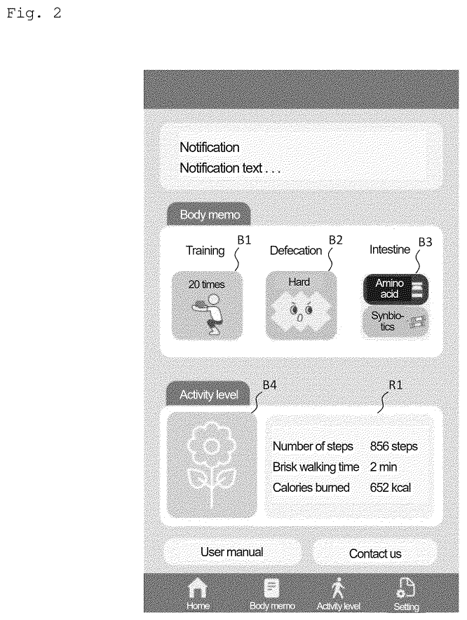

FIG. 2 shows an example of the home screen of the recording application. Buttons B1, B2, and B3 on the home screen are links to transition to screens for recording the patient's strength training status, the patient's bowel movements, and nutrition taken by the patient, respectively.

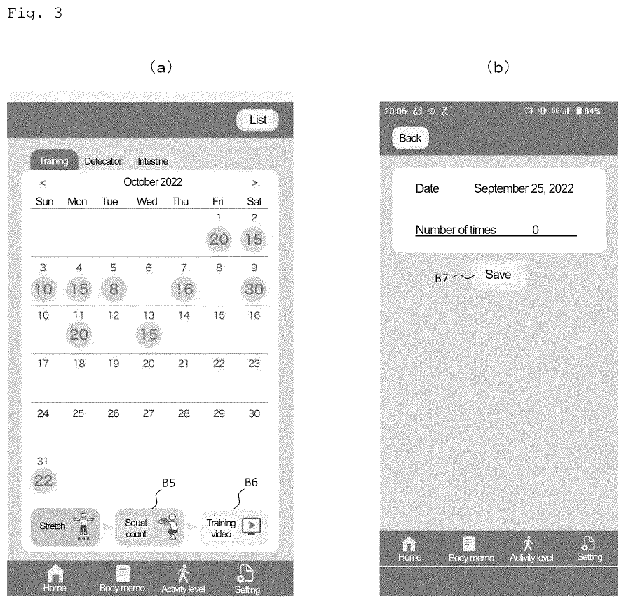

When the button B1 is tapped, the screen changes to that shown in FIG. 3 (a). This screen shows a list of the patient's strength training statuses recorded during the waiting period for surgery (e.g., 6 weeks). In this embodiment, the number of squats is recorded as the strength training status. To record a new strength training status, a date on the calendar is tapped, the strength training status for that day is entered on the screen shown in FIG. 3 (b), and the “Save” button B7 is tapped. As a result, the strength training status recording unit 21 records the patient's strength training status.

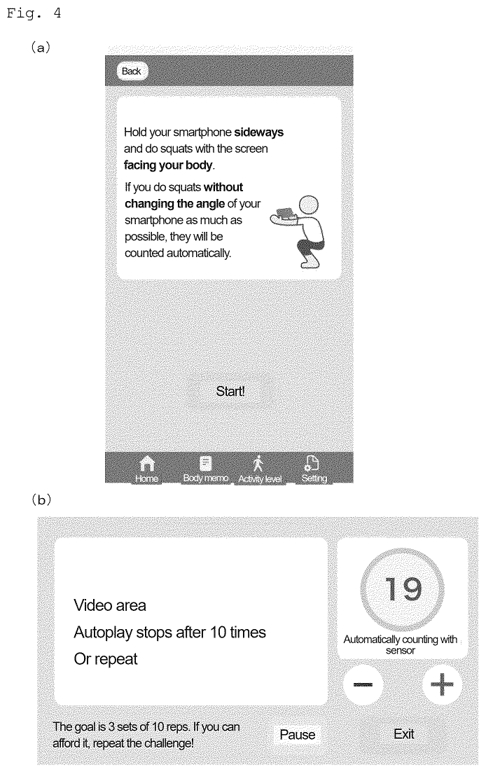

The screen changes to that shown in FIG. 4 (a) by tapping the “Squat count” button B5 in FIG. 3 (a), and the screen changes to that shown in FIG. 4 (b) by tapping the “Start” button B8 in FIG. 4 (a). On this screen, a reference video for strength training is played in the area indicated by “Video area,” and by performing strength training in accordance with the video, the number of repetitions of strength training is measured by a gyro sensor. In this way, the data recorded in the strength training status recording unit 21 can also be automatically input using the gyro sensor.

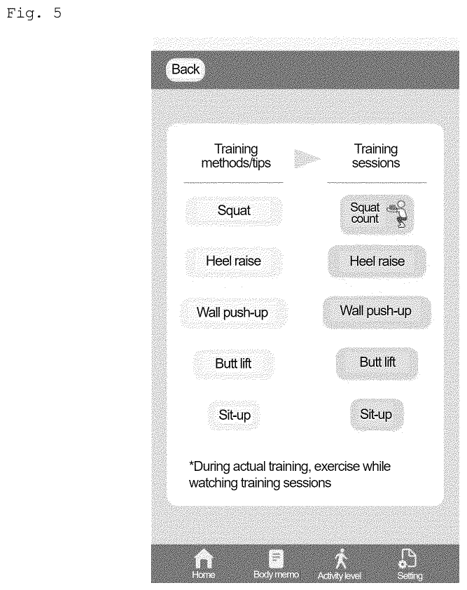

The screen changes to that shown in FIG. 5 by tapping the “Training video” button B6 in FIG. 3 (a). On this screen, the patient can view videos of training methods and tips for each strength training, including those other than squats, as well as videos of training sessions, and the patient can perform strength training while watching the videos. In this embodiment, the number of squats is recorded as the strength training status; however, it is possible to appropriately set the type of strength training to be recorded in the strength training status recording unit 21 in accordance with the prehabilitation program.

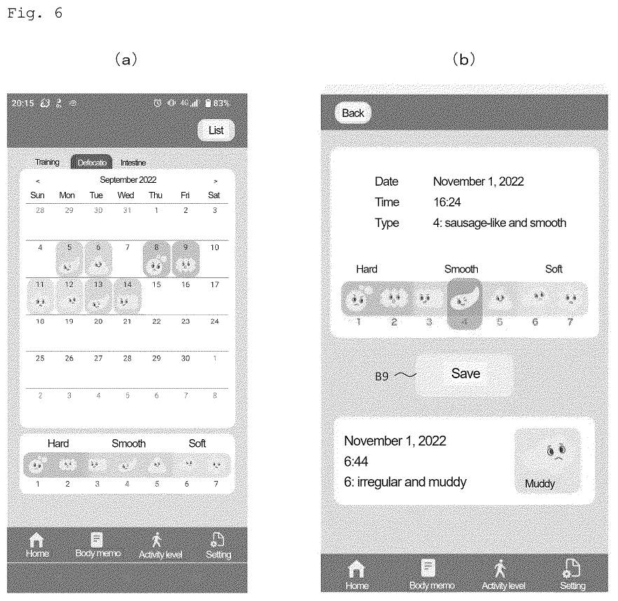

When the button B2 is tapped on the home screen shown in FIG. 2, the screen changes to that shown in FIG. 6 (a). This screen shows a list of the patient's bowel movements recorded during the waiting period for surgery (e.g., 6 weeks). In this embodiment, stool consistency is recorded on a seven-point scale as the bowel movements. To record a new bowel movement, a date on the calendar or the legend indicating the stool consistency at the bottom of the screen is tapped, the time of defecation on that day is entered and the bowel movement is selected on the screen shown in FIG. 6 (b), and the “Save” button B9 is tapped. As a result, the bowel movement recording unit 22 records the patient's bowel movement.

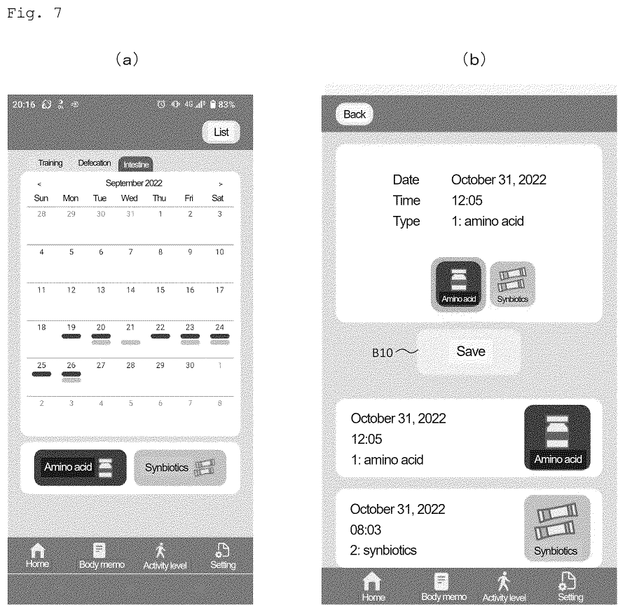

When the button B3 is tapped on the home screen shown in FIG. 2, the screen changes to that shown in FIG. 7 (a). This screen shows a list of nutrition taken by the patient during the waiting period for surgery (e.g., 6 weeks). In this embodiment, the nutrition refers to amino acids and synbiotics. Synbiotics as mentioned herein refer to a combination of probiotics and prebiotics. Probiotics refer to lactic acid bacteria, bifidobacteria, and other live microorganisms that have a beneficial effect on humans by improving the balance of intestinal flora, as well as foods, beverages, and medicines that contain them. Prebiotics refer to oligosaccharides, dietary fiber, and other substances that can be selectively utilized by beneficial microorganisms in the intestines, including probiotics. When the patient takes at least one of these nutrients, a date on the calendar or the legend indicating the nutrition taken at the bottom of the screen is tapped, the time of nutrition intake is entered and the type of nutrition is selected on the screen shown in FIG. 7 (b), and the “Save” button B10 is tapped. As a result, the nutrition recording unit 23 records the nutrition taken by the patient.

The activity level recording unit 24 records the patient's activity level at all times while the recording application is running. In this embodiment, the activity level includes the patient's steps, brisk walking time, and calories burned. The steps are measured by a gyro sensor built into the patient terminal 2. The recorded activity level is displayed in an activity level display region R1 on the home screen shown in FIG. 2. The activity level display region R1 displays icon B4, whose image changes according to the patient's steps or brisk walking time. In this embodiment, it is set to display the second stage when achieving 5,000 steps or 10 minutes of brisk walking, and the third stage when achieving 8,000 steps or 20 minutes of brisk walking.

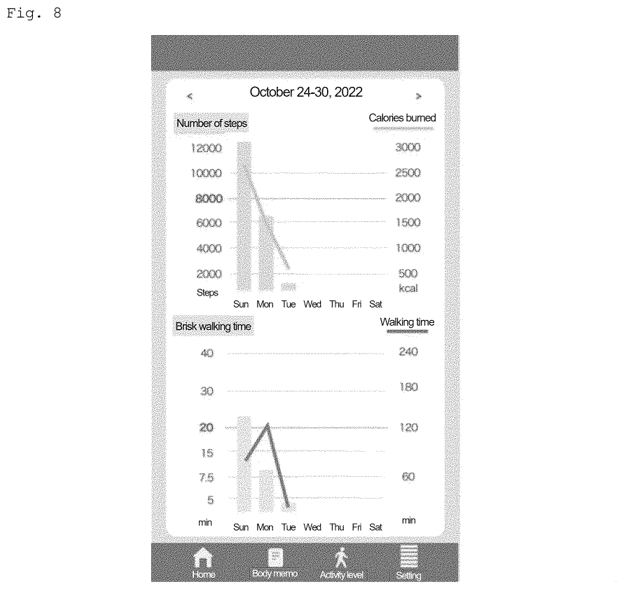

When the icon B4 or the activity level display region R1 is tapped on the home screen, the screen changes to that shown in FIG. 8. This screen shows graphs of the patient's steps and brisk walking time recorded in a week.

The patient terminal 2 may be a general-purpose computer. In this case, the patient's activity level is measured using a separate sensor terminal, such as a pedometer or a wearable device.

The patient's strength training status, patient's bowel movements, nutrition taken by the patient, and patient's activity level recorded in the patient terminal 2 as described above are transmitted as preoperative information D1 to the assistance device 3.

### Assistance Device

The assistance device 3 is located in the cloud and can be composed of a general-purpose computer. The hardware configuration of the assistance device 3 includes a processor, such as CPU or GPU (not shown), a main storage device, such as DRAM or SRAM (not shown), and a secondary storage device 30, such as HDD or SSD. The secondary storage device 30 stores programs, such as an assistance program P1, for operating the assistance device 3, and data.

The assistance device 3 comprises, as function blocks, an acquisition unit 31, a prediction unit 32, an output unit 33, and an alert unit 34. These function blocks can be realized in software by the processor of the assistance device 3. In this case, the processor can read the assistance program P1 stored in the secondary storage device 30 into the main storage device and execute it to realize each of the above units. The assistance program P1 may be downloaded to the assistance device 3 via a communication network, such as the internet, or may be installed on the assistance device 3 via a computer-readable non-transitory recording medium, such as a CD-RCM on which the assistance program P1 is recorded.

The acquisition unit 31 acquires the preoperative information D1 transmitted from the patient terminal 2. In this embodiment, the acquisition unit 31 further stores the preoperative information D1 in the secondary storage device 30.

The prediction unit 32 predicts the patient's postoperative course based on the preoperative information D1. The prediction results are not particularly limited as long as they are useful to determine the postoperative prognostic risk. Examples include the length of hospital stay after surgery and the possibility of complications. Alternatively, preoperative and postoperative medical interventions based on the prognostic risk, and the possibility of surgery may be used as the prediction results.

Further, the prediction unit 32 may perform prediction using, for example, an artificial intelligence (AI) algorithm that uses machine learning of the results of clinical research, or may perform prediction using a rule-based algorithm. In this embodiment, in particular, the preoperative information D1 includes at least one of nutrition taken by the patient and the patient's strength training status, and the nutrition contains at least one of amino acids, probiotics, and prebiotics; thus, the postoperative course can be accurately predicted.

The output unit 33 outputs the prediction results of the prediction unit 32. In this embodiment, the output unit 33 transmits the prediction results to the medical personnel terminal 4.

The alert unit 34 issues a warning to a patient whose postoperative course prediction results are not good even though the scheduled surgery date is approaching. In this embodiment, the alert unit 34 transmits messages to the patient terminal 2 to alert the patient to exercise and take in more nutrition. These messages etc. are displayed, for example, on the notification screen in FIG. 2.

### Medical Personnel Terminal

The medical personnel terminal 4 is a terminal managed by the doctor in charge of the patient's surgery, and can be composed of a general-purpose computer. The medical personnel terminal 4 comprises, as function blocks, an acquisition unit 41, a prediction instruction unit 42, and a prediction result display unit 43. These function blocks can be realized in software by the processor of the medical personnel terminal 4.

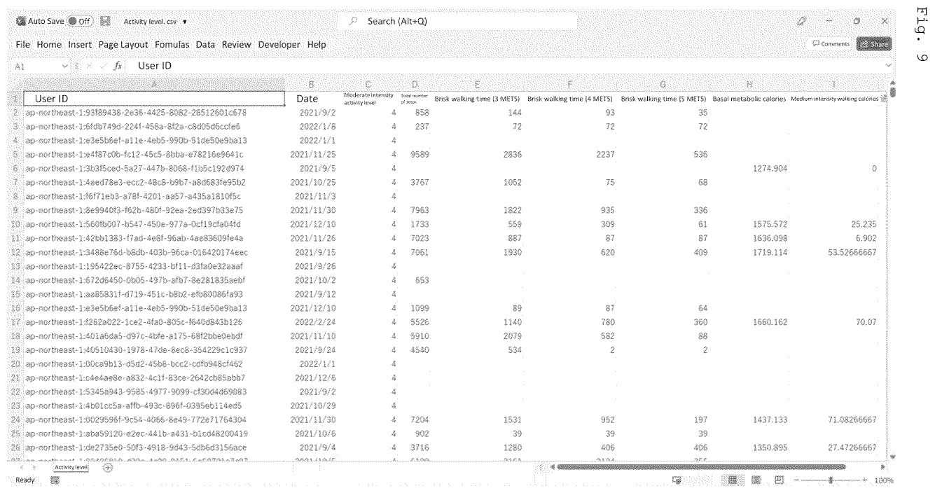

The acquisition unit 41 acquires the preoperative information D1 stored in the secondary storage device 30 of the assistance device 3. FIG. 9 shows an example of the preoperative information D1 displayed on the display of the medical personnel terminal 4. In this example, the activity level of each patient is shown. For important indicators, an alert may be notified to the user (physician) by highlighting, for example.

In this embodiment, the personal data of each patient (age, sex, height, and weight) may be displayed together with the preoperative information D1. Further, information filtered from the preoperative information D1 based on the patient group and time period may also be displayed.

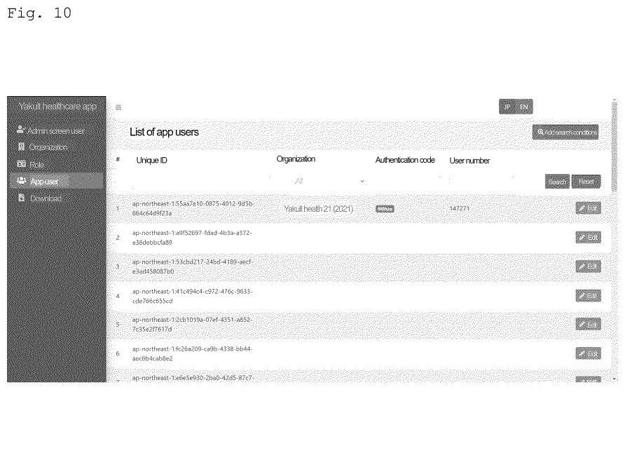

The prediction instruction unit 42 instructs the prediction unit 32 of the assistance device 3 to predict the patient's postoperative course specified by the user depending on the user (doctor)'s operation. FIG. 10 shows an example of a patient list screen displayed on the display of the medical personnel terminal 4. The user can specify a patient from the list screen, and further can give a prediction instruction by performing a certain operation. As a result, the prediction instruction is transmitted from the prediction instruction unit 42 to the assistance device 3, and the prediction unit 32 of the assistance device 3 reads information about the specified patient from the preoperative information D1 stored in the secondary storage device 30, and predicts the postoperative course of the patient based on the information. Thereafter, the output unit 33 transmits the prediction results to the medical personnel terminal 4.

The prediction result display unit 43 displays the prediction results transmitted from the output unit 33 on the display of the medical personnel terminal 4. The user can appropriately determine whether to perform surgery on the patient by referring to the prediction results.

### Processing Steps

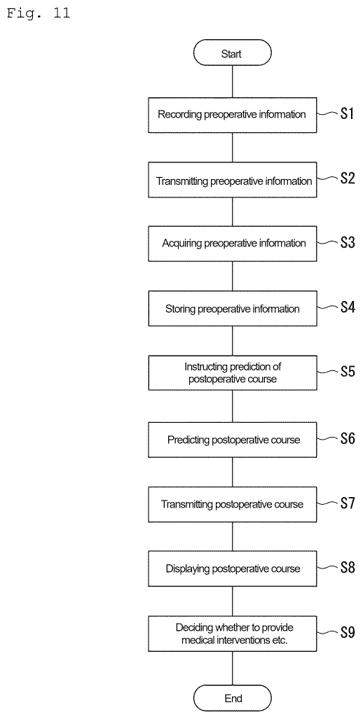

FIG. 11 is a flowchart showing the processing steps of a method for assisting prehabilitation for a patient.

In step S1 (recording step), the patient terminal 2 records preoperative information. In this embodiment, the strength training status recording unit 21, bowel movement recording unit 22, nutrition recording unit 23, and activity level recording unit 24 of the patient terminal 2 record the preoperative information.

In step S2 (transmission step), the preoperative information D1 is transmitted from the patient terminal 2 to the assistance device 3. The timing of transmission is not particularly limited, and may be, for example, every day or on a specific day of the week.

In step S3 (acquisition step), the acquisition unit 31 of the assistance device 3 acquires the preoperative information D1 transmitted from the patient terminal 2.

In step S4, the acquisition unit 31 stores the preoperative information D1 in the secondary storage device 30.

In step S5, depending on the user's operation of the medical personnel terminal 4, the prediction instruction unit 42 instructs the assistance device 3 to predict the patient's postoperative course.

In step S6 (prediction step), the prediction unit 32 of the assistance device 3 predicts the patient's postoperative course based on the preoperative information D1.

In step S7, the output unit 33 transits the prediction results to the medical personnel terminal 4.

In step S8, the prediction result display unit 43 of the medical personnel terminal 4 displays the prediction results on the display of the medical personnel terminal 4.

In step S9, the user of the medical personnel terminal 4 determines whether to perform preoperative and postoperative medical interventions or surgery based on the patient's prognostic risk by referring to the prediction results.

### SUMMARY

As described above, in the above embodiment, the patient's postoperative course is predicted based on the preoperative information D1 including at least one of nutrition taken by the patient and the patient's strength training status, and the nutrition contains at least one of amino acids, probiotics, and prebiotics. Therefore, it is possible to accurately predict the postoperative course, and to appropriately assist prehabilitation. Further, in the above embodiment, the preoperative information D1 is recorded using the patient terminal 2 managed by the patient, which can reduce the burden on doctors in prehabilitation.

In the above embodiment, the assistance device 3 in the cloud predicts the patient's postoperative course; however, the medical personnel terminal 4 or the patient terminal 2 may have at least some of the functions of the assistance device 3. Further, in the above embodiment, the user of the medical personnel terminal 4 determines whether to perform preoperative and postoperative medical interventions or surgery based on the prognostic risk by referring to the prediction results of the assistance device 3; however, whether to perform preoperative and postoperative medical interventions or surgery may be determined by referring to the preoperative information D1. In this case, it is not necessary to predict the postoperative course by the assistance device 3.

The preoperative information D1 may also include the patient's sleep state.

### EXAMPLES

Examples of the present invention are described below. The present invention is not limited to the following Examples.

### Verification Method

In this Example, whether the assistance method according to the present invention could appropriately assist prehabilitation was verified. The subjects were seven patients aged 20 years or older who had undergone highly invasive hepatobiliary-pancreatic surgery, subtotal esophagectomy, or similar highly invasive gastrointestinal surgery for malignant tumors or similar diseases at the Department of Gastrointestinal Surgery, Nagoya University Hospital. Before carrying out prehabilitation, the subjects were asked to install the assistance program P1 (prehabilitation health care application, also known as “Preha” (registered trademark)) according to the above embodiment on their smartphones, and through Preha, they received guidance in the form of narrated videos from rehabilitation department physical therapists and performed preoperative exercise therapy.

There was a waiting period of 2 to 4 weeks before surgery, during which the subjects underwent prehabilitation at home. In addition, all subjects were instructed to continue taking an amino acid preparation for muscle mass enhancement (Amino Perfect Multi Support (registered trademark); four packets are taken twice a day) and a synbiotic preparation for improving the intestinal environment (Synprotec (registered trademark); two probiotic packets and two prebiotic packets are taken per day). Daily activity levels were recorded on Preha, and the subjects were also asked to periodically enter their own data on the bowel status, the intake status of the amino acid preparation and synbiotic preparation, and the Edmonton assessment items. The subjects' preoperative waiting period and postoperative hospital stay were approximately two months in total.

The following primary endpoints a to c were monitored and evaluated as indicators of prehabilitation compliance.
a) Average daily steps during the preoperative waiting period
b) The percentage of the number of days during the preoperative waiting period when the subject walked 5,000 steps or more per day out of the total number of days during the waiting period
c) Average time spent briskly walking per day

In addition, as secondary endpoints, the following d and e were evaluated for exercise capacity, muscle mass, and fat mass measurement at the time of initial hospitalization (hospitalization for examinations required for surgery) and before surgery.
d) Assessment of exercise capacity

Measurement of 6-minute walking distance, grip strength, etc.
e) Assessment of body composition

Measurement of muscle mass, body fat mass, and bone mass using a body composition analyzer (InBody)

Measurement of psoas major muscle area, average CT value, visceral fat mass, and subcutaneous fat mass at the third lumbar vertebra level using CT images

Further, the following information about the subjects was collected from their medical records.
Preoperative factors: age, sex, weight, BMI, underlying disease, Charlson comorbidity index, and stage of malignant tumors
Intraoperative factors: surgical procedure, operative time, intraoperative blood loss, and the presence or absence of intraoperative allogeneic blood transfusion
Postoperative factors: the presence or absence of major complications (Clavien-Dindo grade ≥3) that occurred during the postoperative hospital stay, the type of complications, and the length of postoperative hospital stay

In addition, this hospital also uses a method called GOLD-NUPP as one of the prehabilitation techniques. GOLD-NUPP is a method in which doctors actively intervene in prehabilitation, such as by defining detailed goals for the prehabilitation program together with doctors. Although this requires a lot of work from doctors, it tends to result in a high rate of compliance with prehabilitation. The evaluation results of 71 patients (control group) who had previously undergone prehabilitation using GOLD-NUPP and for whom evaluation results could be collected were used as a Comparative Example.

The surgical procedure, operative time (mean values), and blood loss (median values) performed on the subjects of the Example (Preha group) and the control group of the Comparative Example (GOLD-NUPP group) were as shown in Table 1. The numbers in parentheses for the operative time are standard deviations, and the numbers in parentheses for the blood loss are interquartile ranges (IQR).

TABLE 1

| | | Example | | Comparative Example | |
|---|---|---|---|---|---|
| Surgical | Massive hepatectomy | 4 | (57) | 31 | (47) |
| procedure | Pancreaticoduodenectomy | 3 | (43) | 28 | (42) |
| n (%) | Hepatopancreatoduodenectomy | 0 | (0) | 5 | (8) |
| | Distal pancreatectomy with | 0 | (0) | 2 | (3) |
| | celiac axis resection | | | | |

| | | | | |
|---|---|---|---|---|
| Operative time (min) | 777 | (104) | 593 | (175) |
| Blood loss (mL) | 829 | (728-1117) | 846 | (602-1408) |

The evaluation results of the subjects of the Example and the control group of the Comparative Example were compared using statistical methods. Specifically, the primary endpoints, prehabilitation compliance rates (a to c), were compared with data from historical controls. For the secondary endpoints (d to e), values before and after the introduction of prehabilitation were compared.

### Verification Results

Table 2 shows the average number of steps per day during the preoperative waiting period for the subjects of the Example and the control group of the Comparative Example, the number and percentage of subjects who walked an average of 5,000 steps or more per day during the preoperative waiting period, and the average time spent briskly walking per day. The numbers in parentheses for the average daily steps and average brisk walking time are interquartile ranges (IQR).

TABLE 2

| | Example | | Comparative Example | |
|---|---|---|---|---|
| Average number of steps per day (steps/day) | 5770 | (4513-12307) | 7114 | (5239-10018) |
| Number n of subjects who walked an average of | 4 | (57) | 51 | (72) |
| 5,000 steps or more per day (percentage (%)) | | | | |
| Average brisk walking time (min) | 33.4 | (20.6-68.9) | 10.6 | (1.8-28.6) |

The average number of steps per day and the percentage of subjects who walked an average of 5,000 steps or more per day were slightly lower in the Example than in the Comparative Example; however, the average brisk walking time in the Examples was higher than that in the Comparative Example.

Table 3 shows the 6-minute walking distance (mean values), the amount of change (mean values), and grip strength of the subjects of the Example and the control group of the Comparative Example before performing prehabilitation (Before prehabilitation) and after performing prehabilitation (Immediately before surgery). The numbers in parentheses are standard deviations.

TABLE 3

| | Example | | Comparative Example | |
|---|---|---|---|---|
| | Before | Immediately | Before | Immediately |
| | prehabilitation | before surgery | prehabilitation | before surgery |
| 6-Minute walking | 507.6 (53.4) | 561.0 (41.5) | 515.2 (83.7) | 546.5 (87.3) |
| distance (m) | | | | |
| Amount of change (m) | 53.4 (66.5) | | 31.3 (54.8) | |
| Grip strength (kg) | 27.3 (7.9) | 28.4 (7.0) | — | — |

Thus, the evaluation results of physical ability in the Example were better than those in the Comparative Example.

Table 4 shows the weight (mean values), total skeletal muscle mass (mean values), and total fat mass (mean values) of the subjects of the Example and the control group of the Comparative Example before and after prehabilitation. The numbers in parentheses are standard deviations.

TABLE 4

| | Example | | | | Comparative Example | | | |
|---|---|---|---|---|---|---|---|---|
| | Before | | Immediately | | Before | | Immediately | |
| | prehabilitation | | before surgery | | prehabilitation | | before surgery | |
| Weight (kg) | 58.1 | (9.2) | 60.0 | (8.8) | 61.7 | (9.7) | 61.0 | (9.3) |
| Total skeletal muscle | 8.70 | (1.04) | 9.45 | (1.31) | 9.31 | (1.33) | 9.37 | (1.05) |
| mass (kg/m²) | | | | | | | | |
| Total fat mass (kg/m²) | 16.1 | (6.74) | 11.27 | (6.33) | 5.97 | (2.52) | 5.55 | (2.30) |

There was little change in weight in either the Example or the Comparative Example; however, the increase in total skeletal muscle mass and the decrease in total fat mass were greater in the Example than in the Comparative Example. That is, the body composition evaluation results of the Example were better than those of the Comparative Example.

Table 5 shows the number and percentage (%) of adverse events during the prehabilitation period for the subjects of the Example and the control group of the Comparative Example.

TABLE 5

| | Example | Comparative Example |
|---|---|---|
| Falling down | 0 (0) | 1 (1) |
| Pain (back or leg) | 0 (0) | 4 (6) |
| Deviation of biliary drainage tube | 0 (0) | 1 (1) |
| Vomiting | 0 (0) | 1 (1) |
| Allergy | 0 (0) | 1 (1) |

Thus, some adverse events were observed in the Comparative Example but not in the Example.

Table 6 shows the number of readmissions within 30 days, the number of deaths within 90 days, the number of major complications (Clavien-Dindo grade 3), and the length of postoperative hospital stay (median values) for the subjects of the Example and the control group of the Comparative Example. The numbers in parentheses for the length of postoperative hospital stay are interquartile ranges (IQR).

TABLE 6

| | Example | | Comparative Example | |
|---|---|---|---|---|
| Readmissions within 30 days n (%) | 1 | (13) | 4 | (6) |
| Deaths within 90 days n (%) | 0 | (0) | 2 | (3) |
| Major complications n (%) | 6 | (75) | 40 | (61) |
| Postoperative hospital stay (days) | 38 | (28-49) | 25 | (17-35) |

Thus, the Comparative Example was better than the Example in terms of the number of readmissions within 30 days, the number of major complications, and the length of postoperative hospital stay. However, this is considered to be because, as shown in Table 1, the proportion of massive hepatectomy was higher and the operative time was significantly longer in the Example than in the Comparative Example, suggesting that more invasive surgery may have been performed in the Example. Therefore, it can be said that there is no significant difference in the postoperative course between the Example and the Comparative Example.

### SUMMARY

As described above, the subjects who underwent prehabilitation using the assistance method according to the present invention were able to obtain evaluation results that were roughly equivalent to those of the control group who underwent prehabilitation using GOLD-NUPP, in which doctors actively intervene. Therefore, it was found that the assistance method according to the present invention can appropriately assist prehabilitation and reduce the burden on doctors.

### REFERENCE SIGNS LIST

1. Assistance system
2. Patient terminal
21. Strength training status recording unit
22. Bowel movement recording unit
23. Nutrition recording unit
24. Activity level recording unit
3. Assistance device
30. Secondary storage device
31. Acquisition unit
32. Prediction unit
33. Output unit
34. Alert unit
4. Medical personnel terminal
41. Acquisition unit
42. Prediction instruction unit
43. Prediction result display unit
D1. Preoperative information
P1. Assistance program

1. An assistance method using a computer for assisting prehabilitation for a patient who is scheduled to undergo surgery,
the method comprising an acquisition step of acquiring preoperative information on the patient's condition during a specified period before surgery,
the preoperative information including at least one of nutrition consumed by the patient and the patient's strength training status, and
the nutrition containing at least one of amino acids, probiotics, and prebiotics.

2. The assistance method according to claim 1, wherein the nutrition contains at least one of amino acids and synbiotics.

3. The assistance method according to claim 1, wherein the preoperative information includes the patient's bowel movements.

4. The assistance method according to claim 1, wherein the preoperative information includes the patient's activity level.

5. The assistance method according to claim 4, wherein the activity level includes the patient's steps and calories burned.

6. The assistance method according to claim 1, further comprising a prediction step of predicting the patient's postoperative course based on the preoperative information.

7. The assistance method according to claim 1, further comprising, before the acquisition step:
a recording step of recording the preoperative information using a patient terminal managed by the patient; and
a transmission step of transmitting the preoperative information from the patient terminal to the computer.

8. An assistance program for assisting prehabilitation for a patient who is scheduled to undergo surgery,
the assistance program causing a computer to execute an acquisition step of acquiring preoperative information on the patient's condition during a specified period before surgery,
the preoperative information including at least one of nutrition consumed by the patient and the patient's strength training status, and
the nutrition containing at least one of amino acids, probiotics, and prebiotics.

9. An assistance device for assisting prehabilitation for a patient who is scheduled to undergo surgery,
the assistance device comprising an acquisition unit for acquiring preoperative information on the patient's condition during a specified period before surgery,
the preoperative information including at least one of nutrition consumed by the patient and the patient's strength training status, and
the nutrition containing at least one of amino acids, probiotics, and prebiotics.

10. An assistance system comprising:
the assistance device according to claim 9; and
a patient terminal communicably connected to the assistance device and recording the preoperative information.
